(19) United States
(12) Patent Application Publication
(10) Pub. No.: US 2010/0278730 A1
(43) Pub. Date: Nov. 4, 2010

(54) Non-Invasive Molecular Imaging of Cellular Histone Deacetylase Substrate Using Magnetic Resonance Spectroscopy (MRS) or Positron Emission Tomography (PET)

(76) Inventors: Sabrina Ronen, Orinda, CA (US); Juri Gelovani, Pearland, TX (US); William Tong, Houston, TX (US); Mian Alauddin, Houston, TX (US); Uday Mukhopadhyay, Houston, TX (US); Madhuri Sankaranatayanapillai, Houston, TX (US); Ashutosh Pal, Houston, TX (US)

Correspondence Address: WILLIAMS, MORGAN & AMERSON, 10333 RICHMOND, SUITE 1100, HOUSTON, TX, 77042, US

(21) Appl. No.: 12/595,281

(22) PCT Filed: Apr. 8, 2008
(86) PCT No.: PCT/US08/59620

§ 371 Date: Jul. 14, 2010

Related U.S. Application Data
(60) Provisional application No. 60/923,056, filed on Apr. 12, 2007.

(51) Int. Cl. A61K 51/00; A61B 5/055; C07C 237/28

(52) U.S. Cl. 424/1.65; 424/9.3; 564/155

(57) ABSTRACT

We disclose methods of detecting histone deacetylase activity in a mammal by administering to the mammal a compound comprising at least one atom having a nucleus detectable by magnetic resonance spectroscopy, wherein the compound is a substrate of histone deacetylase; and observing the compound or a cleavage product thereof in at least a portion of the body of the mammal by magnetic resonance spectroscopy (MRS). We also disclose methods of detecting histone deacetylase activity in a mammal by administering to the mammal a compound comprising at least one positron-emission-decaying radioisotope, wherein the compound is a substrate of histone deacetylase; and observing the compound or a cleavage product thereof in at least a portion of the body of the mammal by positron emission tomography (PET). We also disclose compounds useful as histone deacetylase substrates.

This invention was made with United States government support under Grant BC033684, Contract No. W81WH-04-1-0674, “In vivo imaging agent for histone deacetylase,” granted by the United States Department of Defense. The United States government has certain rights in the invention.

### BACKGROUND OF THE INVENTION

The present invention relates generally to the field of treatments for cancer, brain insult, and heart disease. More particularly, it concerns detection of activity of histone deacetylase.

Histone deacetylases (HDACs) regulate gene transcription by deacetylating histone molecules in chromatin. Four classes of HDACs are known: Class I, Zn⁺²-dependent, HDACs 1, 2, 3, and 8; Class II a/b, Zn⁺²-dependent, HDACs 4, 5, 6, 7, 9, and 10; Class III, Zn⁺²-independent NAD-dependent, silent information regulators (sirtuins) 1-8; and Class IV, Zn⁺²-dependent, HDAC 11. Upregulation of HDACs has been implicated in a number of neoplasms, including but not limited to non-Hodgkins lymphoma, Hodgkins lymphoma, leukemia, MDS, pancreatic cancer, colorectal cancer, ovarian cancer, epithelial ovarian cancer, fallopian tube cancer, CML, MPD, AML, liver cancer, mesothelioma, and soft tissue sarcoma.

Histone deacetylase (HDAC) substrates are emerging as a new and exciting class of anti-neoplastic agents. One member of this class, suberoylanilide hydroxamic acid (SAHA), also known as vorinostat and available under the trade name Zolinza® from Merck & Co., Inc., White House Station, N.J., has received U.S. Food and Drug Administration (FDA) approval for treatment of cutaneous T cell lymphoma. Other initial clinical trials have been promising. Treatment with HDAC substrates results in inhibition of cell proliferation and induction of differentiation or apoptosis in cells and tumors. However, treatment can frequently result in tumor stasis and therefore detection of drug molecular action or response to treatment can be difficult.

Histone deacetylases have also been observed to be active in mediating the effects of brain insult, such as stroke or oxidative stress diseases, as well as heart disease. Although inhibition of a histone deacetylase may be effective in treating these disorders, again, detection of drug molecular action or response to treatment can be difficult.

Therefore, a need exists for methods of detecting a histone deacetylase activity in vivo.

### SUMMARY OF THE INVENTION

In one embodiment, the present invention relates to a method of detecting a histone deacetylase activity in a mammal, comprising administering to the mammal a compound comprising at least one atom having a nucleus detectable by magnetic resonance spectroscopy, wherein the compound is a substrate of a histone deacetylase; and observing the compound or a cleavage product thereof in at least a portion of the body of the mammal by magnetic resonance spectroscopy (MRS).

In one embodiment, the present invention relates to a method of detecting a histone deacetylase activity in a mammal, comprising administering to the mammal a compound comprising at least one positron-emission-decaying radioisotope, wherein the compound is a substrate of a histone deacetylase; and observing the compound or a cleavage product thereof in at least a portion of the body of the mammal by positron emission tomography (PET).

In one embodiment, the present invention relates to a histone deacetylase substrate composition, comprising a compound selected from the group consisting of compounds having structure I:

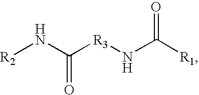

wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -PhC≡C, -triazolyl-(CH₂)ₘX, -Ph-triazolyl-(CH₂)ₘX, -Ph(CH₂)ₘX, -Ph(CH₂)ₘ, -PhN(CH₃)₂, -Ph(CH₂)ₘ, -PhN⁺(CH₃)₃, -Ph(CH₂)ₘNO₂, -Ph(CH₂)ₘ, -Ph C≡C, -triazolyl-(CH₂)ₘX, -Ph-triazolyl-(CH₂)ₘX , -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; and n is an integer from 0 to 10, inclusive; and salts and esters thereof.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A. T₂-weighted RARE image (TE=45 ms, TR=2 s) of PC-3 tumor. The sphere in the image is the external reference observed in the spectra. B. ¹⁹F spectrum of PC-3 tumor (5-minute acquisition time, 300 scans, TR=1 s) acquired 10 minutes following BLT injection i.p. C. Temporal evolution of tumor BLT levels indicating ˜25% decrease in BLT during the first 50 min following BLT injection (for analysis, spectra were added resulting in 10-min time points). D. ³¹p MR spectrum of the same PC-3 tumor (30-minute acquisition time, 900 scans, TR=2 s). E. ¹H spectrum of different tumor (5-minute acquisition, 96 scans, TR=3 s).

FIG. 2. BLT levels normalized to tumor size as determined from the fluorine MR spectra of PC-3 tumors prior to and following 1 week of treatment with 50 mg/kg SAHA in DMSO daily i.p (treatment group) and treatment with carrier DMSO only (control group). Consistent with previous findings in cells, BLT levels were higher in tumors treated with the HDAC substrate SAHA relative to control group as well as tumors prior to treatment.

FIG. 3. ¹⁹F MRS of BLT detects HDAC inhibition prior to effect on tumor size.

FIG. 4. ³¹P MRS and ¹H MRS show a transient increase in phosphomonoesters and choline-containing metabolites following SAHA treatment.

FIG. 5. A, In-vitro uptake study of [¹⁸F]-FAHA in MB435 cell line with/without SAHA. (10 μM of SAHA were treated 1 hr prior to adding [¹⁸F]-FAHA as an substrate). B, In-vitro uptake study of ¹⁴C-FAc in MDA-MB435 cell line.

FIG. 6, photograph of a subject rat of Examples 5-6, with location of the brain roughly indicated by the red dashed oval and location of the tumor indicated by the green dashed oval.

FIG. 7. A & B, qualification of [¹⁸F]-FAHA in rat brain and tumor of subject rat as shown in FIG. 6. FIG. 7A also shows the color legend (the injected dose/gram represented by various colors in the Figures). C & D, quantification of [¹⁸F]-FAHA in rat brain, tumor, and other tissues.

FIG. 8, A & B, quantification of [¹⁸F]-FAHA in rat tumor and muscle.

FIG. 9. A, B, & C, three timecourses (0-60 min) of views of [¹⁸F]-FAHA imaging by PET in the presence or absence of SAHA. The tumor location is shown by the yellow arrow in the 60 min image.

FIG. 10. A & B, predicted versus observed % ID/mL blood of [¹⁸F]-FAHA in the presence or absence of SAHA as imaged by PET.

FIG. 11. Patlak plot analysis of [¹⁸F]-FAHA distribution in rat of Examples 5-6.

FIG. 12. Plot of radioactivity uptake of [¹⁸F]-FAHA by rat brain in the presence or absence of SAHA as imaged by PET.

FIG. 13. A, B, C, and D, four timecourses (0-60 min) of views of [¹⁸F]-FAHA imaging by PET in the presence or absence of SAHA.

FIG. 14. A-I, transaxial sectional views of [¹⁸F]-FAHA imaging by PET in the presence or absence of SAHA.

FIG. 15 shows a synthesis scheme for 6-acetamido-1-(4-fluoro)-hexanoicanilide.

FIG. 16 shows a synthesis scheme for 6-acetamido-1-(4-[¹⁸F]fluoro)-hexanoicanilide.

FIG. 17 shows a synthesis scheme for 6-trifluoroacetamido-1-(4-[¹⁸F]fluoro)-hexanoicanilide.

FIG. 18 shows another synthesis scheme for 6-trifluoroacetamido-1-(4-[¹⁸F]fluoro)-hexanoicanilide.

FIG. 19 shows a synthesis scheme for 6-acetamido-1-[(2-fluoroethyl)-1H-(1,2,3)triazole-4-yl]-hexanoicanilide.

FIG. 20 shows three synthesis schemes for compounds according to various embodiments of the present invention.

FIG. 21 shows the activity of HDACs 1-11 in the presence of various HDAC substrates and BPS#3, a commercially available reference control substrate.

FIG. 22 shows activity of sirtuins 1-5 on various HDAC substrates, relative to the reference peptide BPS#3.

### DESCRIPTION OF ILLUSTRATIVE EMBODIMENTS

In one embodiment, the present invention relates to a method of detecting a histone deacetylase activity in a mammal, comprising administering to the mammal a compound comprising at least one atom having a nucleus detectable by magnetic resonance spectroscopy, wherein the compound is a substrate of a histone deacetylase; and observing the compound or a cleavage product thereof in at least a portion of the body of the mammal by magnetic resonance spectroscopy (MRS).

Any mammal can be the subject of the method. In one embodiment, the mammal is Homo sapiens or a mammal of economic or aesthetic utility. Examples of such mammals include cattle, horses, sheep, dogs, and cats, among others. In a further embodiment, the mammal is Homo sapiens.

The “histone deacetylase” can be any histone deacetylase (HDAC) or sirtuin. At the present time, eleven HDACs and eight sirtuins are known. The person of ordinary skill in the art will recognize that other histone deacetylases, sirtuins, or both may exist and may be discovered, characterized, or both after the filing date of the present application.

The types of HDACs and their characterization, including their distribution and cell type specific expression, are reviewed by Glaser, et al., Biochem. Pharamacol. 74:659-671 (2007); de Ruijter, et al., Biochem. J. 370:737-749 (2003); Broide, et al., J. Molec. Neurosci. 31:47-58 (2007); and Young, et al., manuscript in preparation.

In the administering step, any compound that contains at least one atom having a nucleus detectable by magnetic resonance spectroscopy can be used. In one embodiment, the compound comprises at least one radioisotope selected from the group consisting of ¹H, ²H, ³H, ¹¹C, ¹³C, ¹⁴C, ¹⁵N, ¹⁵O, ¹⁸F, ¹⁹F, and ³¹P. In a further embodiment, the compound contains at least one ¹H, ³¹P, ¹⁹F, ¹³C, or ¹⁵N atom.

Particularly useful are compounds that meet the following criteria: 1) compounds that are substrates of at least one HDAC; 2) compounds that can cross the cell membrane by non-facilitated diffusion; 3) compounds that are radiolabeled such that the radiolabeled product of an enzymatic reaction mediated by at least one HDAC will be metabolically entrapped or temporarily retained inside the cell, whereas the intact parent compound is rapidly cleared from the cell; and 4) the magnitude of accumulation of the radiolabeled product reflects the level of expression and activity of the at least one HDAC in a cell, tissue, or organ.

In one embodiment, the compound is a cleavable substrate of the histone deacetylase. In one embodiment, the compound is one with a magnetic resonance spectrum different in one or more parameters, such as peak height or peak location, from the products(s) produced by cleavage of the compound by the histone deacetylase.

In one embodiment, the compound used in the method is Boc-lysine trifluoroacetic acid (BLT) or a salt or ester thereof. Although BLT has two products when cleaved by a histone deacetylase, the only product to be detectable by magnetic resonance spectroscopy is trifluoroacetic acid (TFA).

In one embodiment, the compound is selected from the group consisting of compounds having structure I:

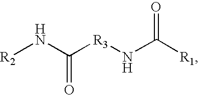

wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -PhC≡C, -triazolyl-(CH₂)ₘX, -Ph-triazolyl-(CH₂)ₘX, -Ph(CH₂)ₘ(CH₂)ₘX, -Ph(CH₂)ₘN(CH₃)₂, -Ph(CH₂)ₘ, -Ph N⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; and n is an integer from 0 to 10, inclusive; and salts and esters thereof.

The person of ordinary skill in the art will understand that “-Ph” refers to a phenyl moiety.

In a further embodiment, the compound is selected from the group consisting of compounds having structure I, wherein R₁ is selected from the group consisting of —CH₃, —CH₂X, —CHX₂, —CX₃, —(CH₂)ₘCH₃, and —CH₂(CH₃)₂; R₂ is -Ph; m is an integer from 1 to 2, inclusive; and n is 5.

In a further embodiment, the compound having structure I as defined above is selected from the group consisting of 6-(fluoroacetamido)-1-hexanoicanilide (FAHA), 6-(trifluoroacetamido)-1-hexanoicanilide (3FAHA), 6-(acetamido)-1-hexanoicanilide (AHA), 6-(methylacetamido)-1-hexanoicanilide (EAHA), 6-(ethylacetamido)-1-hexanoicanilide (PAHA), 6-(isopropylacetamido)-1-hexanoicanilide (Iso-PAHA), 6-(phenylacetamido)-1-hexanoicanilide (PhAHA), 6-(bromoacetamido)-1-hexanoicanilide (BrAHA), 6-(1-bromo-1-difluoroacetamido)-1-hexanoicanilide (Br2FAHA), 6-acetamido-1-[(1-ethyl(2-fluoro)piperidenyl-4-amino)]-hexanamide (FEPIAHA), 6-acetamido-1-[(2-fluoroethyl)-1H-(1,2,3)triazole-4-yl)]-hexanamide (FETrAHA), 6-acetamido-1-[piperidenyl-(4-amino)]-hexanamide (PIAHA), 6-(trifluoroacetamido)-1-(4-fluoro)hexanoicanilide (F—F3FAHA), 6-(1-bromo-1-difluoroacetamido)-1-(4-fluoro)hexanoicanilide (F—Br2FAHA), 6-acetamido-1-(4-fluoro)-hexanoicanilide, 6-acetamido-1-[4-(N,N-dimethylamino)]-hexanoicanilide, 6-acetamido-1-(4-trimethylammoniumtriflate)-hexanoicanilide, 6-acetamido-1-(4-nitro)-hexanoicanilide, 6-trifluoroacetamido-1-[4-(N,N-dimethylamino)]-hexanoicanilide, trifluoroacetamido-1-(4-trimethylammoniumtriflate)-hexanoicanilide, 6-acetamido-1-[4-(ethynyl]]-hexanoicanilide, 6-acetamido-1-[(2-fluoroethyl)-1H-(1,2,3)triazole-4-yl]]-hexanoicanilide, N-[(4-acetylamino)benzyl]acetamide, N-{[(4-(2-fluoroacetyl)amino)benzyl]}-2-fluoroacetamide, N-{[(4-(2-fluoroacetyl)amino)benzyl]}-acetamide, N-{[(4-acetylamino)benzyl]}-2-fluoroacetamide, N-{[(4-(2-bromooacetyl)amino)benzyl]}-2-fluoroacetamide, N-{[(4-(2-bromoacetyl)amino)benzyl]}-acetamide, and salts and esters thereof.

In one embodiment, the compound is selected from the group consisting of Boc-lysine trifluoroacetic acid (BLT); compounds having structure I as defined above; and salts and esters thereof.

Particular compounds identified above may be especially useful in detecting the activity of particular HDACs. In one embodiment, the pairing of the histone deacetylase and the histone deacetylase substrate is selected from the group consisting of HDAC 1 and FAHA; HDAC-4 and FAHA; HDAC-5 and FAHA; HDAC-6 and FAHA; HDAC-8 and FAHA; HDAC-9 and FAHA; HDAC-4 and 3FAHA; HDAC-5 and 3FAHA; HDAC-7 and 3FAHA; HDAC-8 and 3FAHA; HDAC-9 and 3FAHA; HDAC-11 and 3FAHA; HDAC-3 and EAHA; sirtuin-2 and EAHA; HDAC-3 and PAHA; sirtuin-1 and PAHA; sirtuin-2 and PAHA; sirtuin-3 and PAHA; sirtuin-4 and PAHA; sirtuin-5 and PAHA; sirtuin-1 and Iso-PAHA; sirtuin-2 and Iso-PAHA; sirtuin-3 and Iso-PAHA; sirtuin-4 and Iso-PAHA; and iso-PAHA; sirtuin-1 and PhAHA; sirtuin-2 and PhAHA; sirtuin-3 and PhAHA; sirtuin-4 and PhAHA; and sirtuin-5 and PhAHA.

Particularly, we qualitatively rank the specificity of various HDACs for FAHA as HDAC-9>HDAC-5>HDAC-8>HDAC-4>HDAC-6>HDAC-1. We qualitatively rank the specificity of various HDACs for 3FAHA as HDAC-9>HDAC-7>HDAC-5>HDAC-8>HDAC-4>HDAC-11. We qualitatively rank the specificity of various HDACs for PAHA as Sirt-2>Sirt-5>Sirt-4>Sirt-3>Sirt-1 >>HDAC-3. We qualitatively rank the specificity of various sirtuins for Iso-PAHA as Sirt-2>Sirt-5>Sirt-4>Sirt-3>Sirt-1. We qualitatively rank the specificity of various sirtuins for PhAHA as Sirt-2 >>Sirt-3>Sirt-4>Sirt-5>Sirt-1.

In the observing step, magnetic resonance spectroscopy (MRS) is used to observe the compound in the mammal. If in vivo a histone deacetylase has activity, the intensity of an MRS signal derived from the compound that contains at least one atom having a nucleus detectable by magnetic resonance spectroscopy, if the compound is a cleavable substrate of the histone deacetylase, will be strongest at a first observation timepoint and will be weaker at a second, slightly later observation timepoint, because the histone deacetylase will cleave the compound, reducing its concentration and therefore reducing its MRS signal intensity. (“Slightly later” as used in this context means within about two hours after the first observation timpoint). Reversely, the intensity of an MRS signal derived from a compound's histone deacetylase cleavage product(s) will be weakest at a first observation timepoint and will be stronger at a second, slightly later observation timepoint.

On the other hand, if in vivo a histone deacetylase has no activity, the intensity of an MRS signal derived from the compound that contains at least one atom having a nucleus detectable by magnetic resonance spectroscopy, if the compound is a cleavable substrate of the histone deacetylase, will be essentially unchanged from a first observation timepoint to a second, slightly later observation timepoint, because cleavage of the compound will not take place. (The compound will typically be cleared by the kidneys or processed by other organs on a longer timescale, typically hours). Also, essentially no MRS signal will be observed for histone deacetylase cleavage products of the compound.

If in vivo a histone deacetylase has partial activity relative to a baseline representing full activity, such as can occur if a histone deacetylase substrate is or has been administered to the mammal, some reduction in MRS signal intensity derived from the compound that contains at least one atom having a nucleus detectable by magnetic resonance spectroscopy, if the compound is a cleavable substrate of the histone deacetylase, will be seen within about two hours; but the rate or extent of reduction will be less than that seen when the in vivo histone deacetylase has full activity. The difference in rate or extent of reduction of MRS signal intensity can be determined by comparing the reduction of MRS signal intensity to a baseline. For example, if the partial activity of the histone deacetylase results at least in part from administration of a histone deacetylase substrate to the mammal, the baseline can be determined from the activity of the histone deacetylase on the compound when a histone deacetylase substrate is not administered to the mammal.

Any portion of the body in which the skilled artisan having the benefit of the present disclosure may desire to detect a histone deacetylase activity can be observed in the method. In one embodiment, the portion of the body can be one in which the mammal suffers a tumor. In one embodiment, the portion of the body can be the brain.

In one embodiment of the method, the mammal suffers a tumor in the portion of the body, and the method further involves administering to the mammal a histone deacetylase substrate. The tumor can be prostate cancer, breast cancer, brain cancer, or skin cancer (such as cutaneous T cell lymphoma).

In one embodiment, the mammal suffers heart disease. The heart disease can be any acute or chronic ailment of the heart.

Any histone deacetylase substrate can be used. In one embodiment, the histone deacetylase substrate is suberoylanilide hydroxamic acid (SAHA) or a salt or ester thereof. SAHA is also known as vorinostat and is available under the trade name Zolinza® from Merck & Co., Inc., White House Station, N.J.

In one embodiment, the histone deacetylase substrate is selected from the group consisting of compounds having structure I, as described above, and salts and esters thereof. In a further embodiment, the histone deacetylase substrate is selected from the group consisting of FAHA, 3FAHA, AHA, EAHA, PAHA, Iso-PAHA, PhAHA, BrAHA, Br2FAHA, FEPIAHA, FETrAHA, PIAHA, F—F3FAHA, F—Br2FAHA, 6-acetamido-1-(4-fluoro)-hexanoicanilide, 6-acetamido-1-[4-(N,N-dimethylamino)]-hexanoicanilide, 6-acetamido-1-(4-trimethylanunoniumtriflate)-hexanoicanilide, 6-acetamido-1-(4-nitro)-hexanoicanilide, 6-trifluoroacetamido-1-[4-(N,N-dimethylamino)]-hexanoicanilide, trifluoroacetamido-1-(4-trimethylammoniumtriflate)-hexanoicanilide, 6-acetamido-1-[4-(ethynyl]]-hexanoicanilide, 6-acetamido-1-[(2-fluoroethyl)-1H-(1,2,3)triazole-4-yl]]-hexanoicanilide, N-[(4-acetylamino)benzyl]acetamide, N-{[(4-(2-fluoroacetyl)amino)benzyl]}-2-fluoroacetamide, N-{[(4-(2-fluoroacetyl)amino)benzyl]}-acetamide, N-{[(4-acetylamino)benzyl]}-2-fluoroacetamide, N-{[(4-(2-bromooacetyl)amino)benzyl]}-2-fluoroacetamide, N-{[(4-(2-bromoacetyl)amino)benzyl]}-acetamide, and salts and esters thereof.

In one embodiment, the mammal suffers an insult in the brain, and the method further involves administering to the mammal a histone deacetylase substrate. The insult in the brain can be a stroke or oxidative stress, among others.

In this embodiment, the histone deacetylase substrate is as described above.

In one embodiment, the present invention relates to a method of detecting a histone deacetylase activity in a mammal by administering to the mammal a compound comprising at least one positron-emission-decaying radioisotope, wherein the compound is a substrate of the histone deacetylase; and observing the compound or a cleavage product thereof in at least a portion of the body of the mammal by positron emission tomography (PET).

Any compound comprising at least one positron-emission-decaying radioisotope, wherein the compound is a substrate of the histone deacetylase, can be used in this embodiment of the invention. A “positron-emission-decaying radioisotope” is an isotope that undergoes positive beta decay. Exemplary positron-emission-decaying radioisotopes include, but are not limited to, ¹⁸F, ¹¹C, ¹³N, and ¹⁵O, among others.

In one embodiment, the compound comprising at least one positron-emission-decaying radioisotope used in the method is selected from the group consisting of 6-([¹⁸F]-fluoroacetamide)-1-hexanoicanilide ([¹⁸F]-FAHA) Boc-lysine tri[¹⁸F]fluoroacetic acid ([¹⁸F]BLT), and salts and esters thereof.

In one embodiment, the compound is selected from the group consisting of compounds having structure I, as described above; and salts and esters thereof.

In a further embodiment, the compound having structure I is selected from the group consisting of FAHA, 3FAHA, AHA, EAHA, PAHA, Iso-PAHA, PhAHA, BrAHA, Br2FAHA, FEPIAHA, FETrAHA, PIAHA, F—F3FAHA, F—Br2FAHA, 6-acetamido-1-(4-fluoro)-hexanoicanilide, 6-acetamido-1-[4-(N,N-dimethylamino)]-hexanoicanilide, 6-acetamido-1-(4-trimethylammoniumtriflate)-hexanoicanilide, 6-acetamido-1-(4-nitro)-hexanoicanilide, 6-trifluoroacetamido-1-[4-(N,N-dimethylamino)]-hexanoicanilide, trifluoroacetamido-1-(4-trimethylammoniumtriflate)-hexanoicanilide, 6-acetamido-1-[4-(ethynyl]]-hexanoicanilide, 6-acetamido-1-[(2-fluoroethyl)-1H-(1,2,3)triazole-4-yl]]-hexanoicanilide, N-[(4-acetylamino)benzyl]acetamide, N-{[(4-(2-fluoroacetyl)amino)benzyl]}-2-fluoroacetamide, N-{[(4-(2-fluoroacetyl)amino)benzyl]}-acetamide, N-{[(4-acetylamino)benzyl]}-2-fluoroacetamide, N-{[(4-(2-bromooacetyl)amino)benzyl]}-2-fluoroacetamide, N-{[(4-(2-bromoacetyl)amino)benzyl]}-acetamide, and salts and esters thereof.

In one embodiment, the compound used in the method is selected from the group consisting of compounds having structure I, as described above; Boc-lysine tri[¹⁸F]fluoroacetic acid ([¹⁸F]BLT); and salts and esters thereof.

In the observing step, positron emission tomography (PET) is used to localize the compound. Similar to an embodiment discussed previously, the portion of the mammal's body observed in the method can be one in which the mammal suffers a tumor. In one embodiment, the portion of the body can be the brain.

In one embodiment of the method, the mammal suffers a tumor in the portion of the body, and the method further involves administering to the mammal a histone deacetylase substrate. The tumor can be as described above. The histone deacetylase substrate can be as described. In one embodiment, the histone deacetylase substrate is suberoylanilide hydroxamic acid (SAHA) or a salt or ester thereof.

In one embodiment, the histone deacetylase substrate is selected from the group consisting of compounds having structure I, as described above, and salts and esters thereof. In a further embodiment, the histone deacetylase substrate is selected from the group consisting of FAHA, 3FAHA, AHA, EAHA, PAHA, Iso-PAHA, PhAHA, BrAHA, Br2FAHA, FEPIAHA, FETrAHA, PIAHA, F—F3FAHA, F—Br2FAHA, 6-acetamido-1-(4-fluoro)-hexanoicanilide, 6-acetamido-1-[4-(N,N-dimethylamino)]-hexanoicanilide, 6-acetamido-1-(4-trimethylammoniumtriflate)-hexanoicanilide, 6-acetamido-1-(4-nitro)-hexanoicanilide, 6-trifluoroacetamido-1-[4-(N,N-dimethylamino)]-hexanoicanilide, trifluoroacetamido-1-(4-trimethylammoniumtriflate)-hexanoicanilide, 6-acetamido-1-[4-(ethynyl]]-hexanoicanilide, 6-acetamido-1-[(2-fluoroethyl)-1H-(1,2,3)triazole-4-yl]]-hexanoicanilide, N-[(4-acetylamino)benzyl]acetamide, N-{[(4-(2-fluoroacetyl)amino)benzyl]}-2-fluoroacetamide, N-{[(4-(2-fluoroacetyl)amino)benzyl]}-acetamide, N-{[(4-acetylamino)benzyl]}-2-fluoroacetamide, N-{[(4-(2-bromooacetyl)amino)benzyl]}-2-fluoroacetamide, N-{[(4-(2-bromoacetyl)amino)benzyl]}-acetamide, and salts and esters thereof.

In one embodiment, the mammal suffers an insult in the brain, and the method further involves administering to the mammal a histone deacetylase substrate. The insult in the brain can be as described above. The histone deacetylase substrate can be as described above.

In one embodiment, the present invention relates to a histone deacetylase substrate composition, comprising a compound selected from the group consisting of compounds having structure I, as described above; and salts and esters thereof.

In one embodiment, the compound comprises at least one radioisotope selected from the group consisting of ¹H, ²H, ³H, ¹¹C, ¹³C, ¹⁴C, ¹⁵N, ¹⁵O, ¹⁸F, ¹⁹F, and ³¹P.

In one embodiment, the compound is selected from the group consisting of 6-(fluoroacetamido)-1-hexanoicanilide (FAHA), 6-(trifluoroacetamido)-1-hexanoicanilide (3FAHA), 6-(acetamido)-1-hexanoicanilide (AHA), 6-(methylacetamido)-1-hexanoicanilide (EAHA), 6-(ethylacetamido)-1-hexanoicanilide (PAHA), 6-(isopropylacetamido)-1-hexanoicanilide (Iso-PAHA), 6-(phenylacetamido)-1-hexanoicanilide (PhAHA), 6-(bromoacetamido)-1-hexanoicanilide (BrAHA), 6-(1-bromo-1-difluoroacetamido)-1-hexanoicanilide (Br2FAHA), 6-acetamido-1-[(1-ethyl(2-fluoro)piperidenyl-4-amino)]-hexanamide (FEPIAHA), 6-acetamido-1-[(2-fluoroethyl)-1H-(1,2,3)triazole-4-yl)]-hexanamide (FETrAHA), 6-acetamido-1-[piperidenyl-(4-amino)]-hexanamide (PIAHA), 6-(trifluoroacetamido)-1-(4-fluoro)hexanoicanilide (F—F3FAHA), 6-(1-bromo-1-difluoroacetamido)-1-(4-fluoro)hexanoicanilide (F—Br2FAHA), 6-acetamido-1-(4-fluoro)-hexanoicanilide, 6-acetamido-1-[4-(N,N-dimethylamino)]-hexanoicanilide, 6-acetamido-1-(4-trimethylammoniumtriflate)-hexanoicanilide, 6-acetamido-1-(4-nitro)-hexanoicanilide, 6-trifluoroacetamido-1-[4-(N,N-dimethylamino)]-hexanoicanilide, trifluoroacetamido-1-(4-trimethylammoniumtriflate)-hexanoicanilide, 6-acetamido-1-[4-(ethynyl]]-hexanoicanilide, 6-acetamido-1-[(2-fluoroethyl)-1H-(1,2,3)triazole-4-yl]]-hexanoicanilide, N-[(4-acetylamino)benzyl]acetamide, N-{[(4-(2-fluoroacetyl)amino)benzyl]}-2-fluoroacetamide, N-{[(4-(2-fluoroacetyl)amino)benzyl]}-acetamide, N-{[(4-acetylamino)benzyl]}-2-fluoroacetamide, N-{[(4-(2-bromooacetyl)amino)benzyl]}-2-fluoroacetamide, N-{[(4-(2-bromoacetyl)amino)benzyl]}-acetamide, and salts and esters thereof.

In one embodiment, the histone deacetylase substrate composition further comprises a sterile carrier. In a further embodiment, the sterile carrier is an aqueous saline solution.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Toxicity Study of Boc-Lysine Trifluoroacetic Acid (BLT)

Rationale: BLT must have no detectable toxic effects at levels that result in an MRS-visible signal in vivo if it were to be used as an imaging agent. To verify this, we monitored the toxicity of 100 mg/kg BLT (˜3 mM plasma concentration), which, based on previously published studies of fluorinated drugs, was expected to result in an MRS-detectable signal. (Hamstra, et al., Mol Ther, 10: 916-928, 2004; McSheehy, et al., Cancer Res, 60: 2122-2127, 2000; Gade, et al., Magn Reson Med, 52: 169-173, 2004; Chung, et al., Clin Cancer Res, 10:3863-3870, 2004; Aboagye, et al., Cancer Res, 58: 4075-4078, 1998).

Methods and Results: Four male nude mice were injected intraperitoneally with 100 mg/kg BLT in 40 μl of DMSO and 4 mice were injected with 40 μl of DMSO alone on days 1, 8, and 15 of the study. The mice were monitored daily for changes in weight, skin and hydration status, activity, behavior, feeding, and neurological status. On day 16, blood samples were collected, and blood counts and hepatic and kidney functions assessed (bilirubin, total protein, aspartate aminotransferase, alanine aminotransferase, creatinine, and blood urea nitrogen). At the end of the study, following animal euthanasia, mice were dissected and tissue samples obtained for histology (hematoxylin-eosin staining).

Unpredictably, no indication of significant toxicity was observed in the BLT-treated animals compared with DMSO-treated controls.

### Example 2

### Detectability of BLT, PC and tCho In Vivo

Rationale: To assess the feasibility of the in vivo experiments, it was necessary to determine whether a typical magnetic resonance (MR) system, specifically, a 4.7 T Bruker Biospec MR system, had the sensitivity to detect BLT from the tumor region following intraperitoneal injection of a non-toxic dose of BLT, and that in vivo BLT levels are modulated by HDAC inhibition. It was also necessary to determine whether PC and tCho could be monitored in vivo.

Methods and Results: PC-3 cells (10⁶) were injected into the flank of male nude mice. MR experiments were first performed when tumors were ˜1 cm in diameter. Mice were anesthetized using isoflurane. A 10-mm home-built ¹⁹F surface coil (milled from a polymer-based flexible substrate using a ProtoMat C100/HF (LPKF Laser & Electronics, Wilsonville, Oreg.)) was placed over the tumor, and the animal placed at the center of the magnet. Varacator diodes in the impedance matching circuitry provided a means to match and tune the coil from the console. Following localization, a T₂-weighted image of the tumor was recorded using the RARE sequence (TE=45 ms, TR=2 s) via the ¹⁹F surface coil tuned to ¹H. Localized shimming was performed using FASTMAP and achieved ˜20 Hz line width for the water peak. A localized ¹H spectrum was then acquired using a single-voxel PRESS sequence (TE=21 ms, TR=3 s, 96 scans) with VAPOR water suppression. Following retuning of the coil to ¹⁹F, a ‘baseline’ ¹⁹F spectrum was then acquired using a one-pulse sequence (45° flip angle, 300 scans, TR=1 s). A microsphere filled with 20 mM trifluorotoluene (−63.7 ppm) served as an external reference. 100 mg/kg BLT (in 40 μl DMSO) was then injected i.p. and sequential 5-minute ¹⁹F spectra acquired over 90 minutes. The ¹⁹F surface coil was then replaced with a ³¹P coil and a spectrum acquired from the tumor region using a one-pulse sequence (30° flip angle, 900 scans, TR=2 s). Adequate SNR was achieved within 30 min.

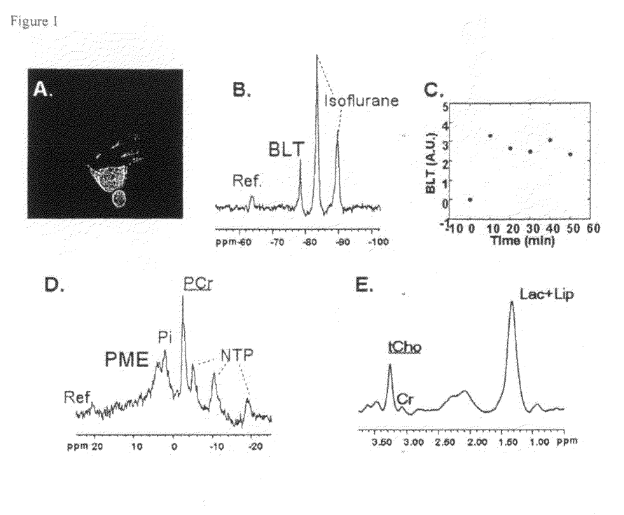

FIG. 1 illustrates the data obtained. It indicates that BLT is clearly detectable in the ¹⁹F spectrum of the tumor with a temporal resolution of 5 minutes. A small shoulder was detected on the BLT peak in some cases, possibly reflecting extracellular TFA (TFA was present in the tumor extract). PC is the principal component of the PME peak (based on extracts) and is clearly detectable in the ³¹P spectrum, and tCho levels can be monitored from the ¹H spectrum.

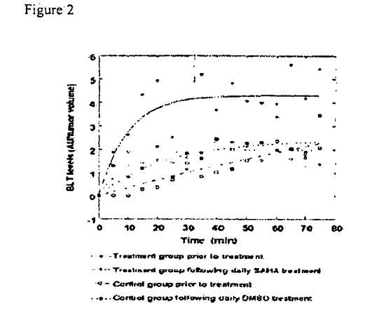

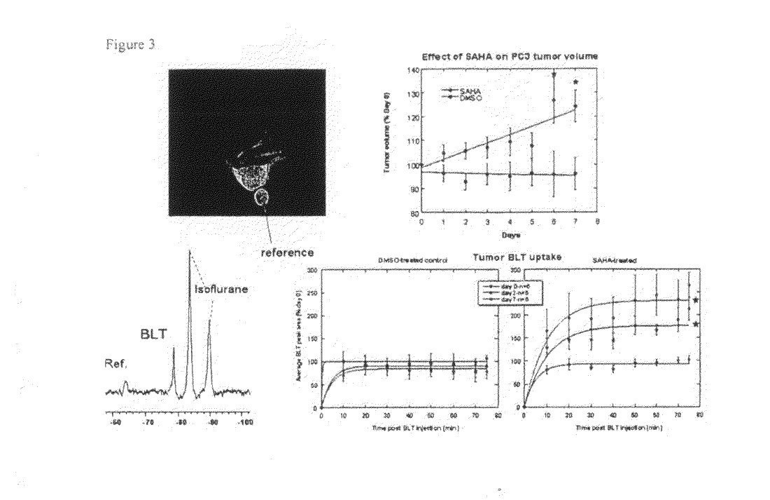

Following this initial study, control mice were treated daily with 40 μl DMSO i.p. and treated mice were treated daily with 50 mg/kg SAHA in 40 μl DMSO i.p. Preliminary data indicate that SAHA treatment resulted in inhibition of tumor growth. Following 1 week of treatment, the MR study was repeated. FIG. 2 illustrates the preliminary results indicating that BLT levels in the tumor in vivo were higher in the SAHA treated tumor compared to control, consistent with the findings in cells.

### Example 3

### In Vivo Detection of Histone Deacetylase Inhibition by MRS

Introduction. Histone deacetylase (HDAC) substrates are emerging as a new and exciting class of anti-neoplastic agents. Initial clinical trials have been promising and treatment with HDAC substrates results in inhibition of cell proliferation and induction of differentiation or apoptosis in cells and tumors. However, treatment can frequently result in tumor stasis and therefore detection of drug molecular action or response to treatment can be difficult. Our goal is to noninvasively monitor inhibition of HDAC at the tumor site. To this end, we have developed a method that uses ¹⁹F magnetic resonance spectroscopy (MRS) to monitor a fluorinated cleavable HDAC substrate (Boc-lysine TFA—BLT). We have shown that BLT levels as determined by MRS can be used to assess HDAC activity in cells. We show here that this method can also be applied in tumors in vivo.

Methods. 5×10⁶ PC3 human prostate cancer cells suspended in matrigel were injected subcutaneously in male CD-1 nude mice (n=5). When an average tumor volume of 0.2 cm³ was reached, mice were separated into 2 groups. The treated group (n=3) was treated daily with 50 mg/kg SAHA intraperitoneally, while the control mice were treated with carrier DMSO. MRS was performed on a 4.7T Biospec (Bruker Biospin, Billerica, Mass.) prior to treatment (day 0) and on days 2 and 7 of treatment, using a 1.5 cm (inner diameter) dual-tuned ¹H/¹⁹F surface coil. Each MR study included T2-weighted RARE imaging, localized ¹H MRS by point-resolved spectroscopy (PRESS-TE/TR=20 ms/3 s) with (100 averages) and without (1 average) water suppression, and ¹⁹F MRS (TR=1 s, 45° flip angle, 300 averages) before and after intraperitoneal injection of 100 mg/kg BLT (Advanced Chem-Tech, KY USA). BLT levels determined by ¹⁹F MRS were normalized to the external reference aaa-trifluorotoluene (TFT) (Sigma-Aldrich Chemical Co., St. Louis, Mo.) in a micro-cell spherical bulb placed at a permanent location relative to the coil and expressed as % of maximum tumor BLT levels in each study. For each mouse and also for average values, a paired t-test of BLT evolution was performed for days 2 and 7 with respect to day 0. ¹H MRS data was analyzed by normalizing the tCho signal either to the total ¹H signal or to the internal water signal.

Results. ¹⁹F MRS of BLT detects HDAC inhibition prior to effect on tumor size (FIG. 3).

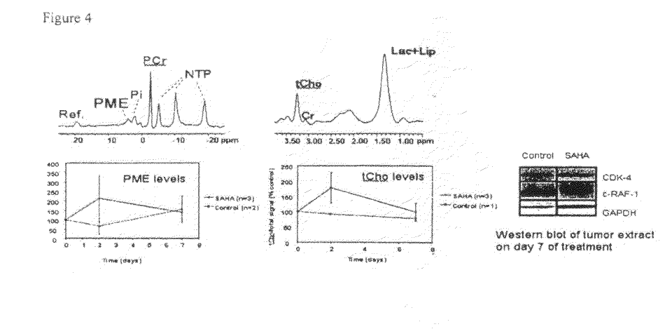

³¹P MRS and ¹H MRS show a transient increase in phosphomonoesters and choline-containing metabolites following SAHA treatment (FIG. 4).

Conclusion. The level of BLT accumulated in a tumor, as determined by noninvasive MRS, gives an early indication of drug action, prior to tumor shrinkage.

### Example 4

### In Vitro Uptake Study in Human Breast Carcinoma Cell Line Using [¹⁸F]-FAHA and Positron Emission Tomography (PET)

MDA-MB435 human breast carcinoma cells were grown into flasks with D-MEM/F-12 medium supplemented with 10% FBS and antibiotics at 37° C. in humidified atmosphere with 5% CO₂. Cells were kept in the log phase proliferative activity. 5×10⁶ of cells in 15 mL of medium were distributed into each tissue culture dish and were incubated at least for 24 hours.

Next day, culture medium was replaced to fresh medium again and was incubated for 3 hours. Then 20 mCi of [¹⁸F]-FAHA in 20 mL of saline was added to each dish followed by incubation for 5, 10, 15, 30, 60, and 120 min. At each time point, cells were scraped and were centrifuged at 3,000 rpm for 1 min. After centrifugation, 100 mL of supernatant medium and cells were weighed and their radioactivity measured using a gamma counter. Then the ratio of radioactivity between 1 gram of cells and 1 gram of medium was calculated.

For an inhibition study, cells were incubated in medium with 10 mM of SAHA from 1 hour before adding [¹⁸F]-FAHA.

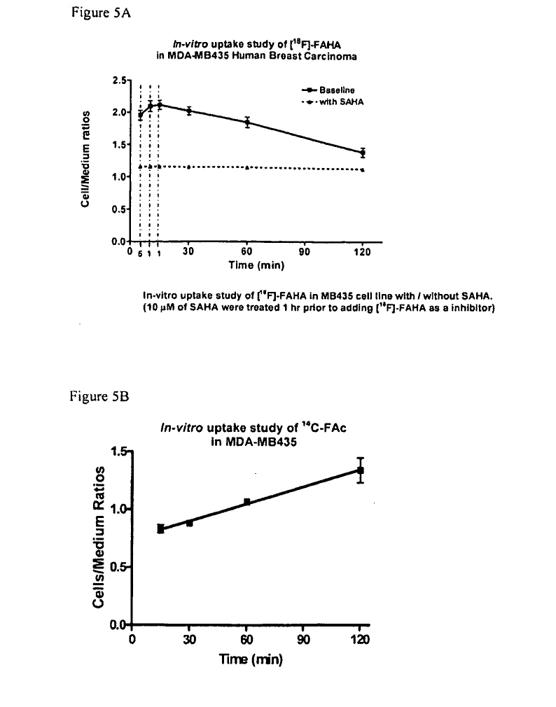

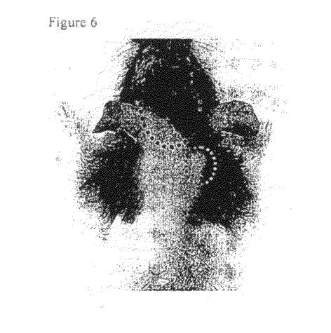

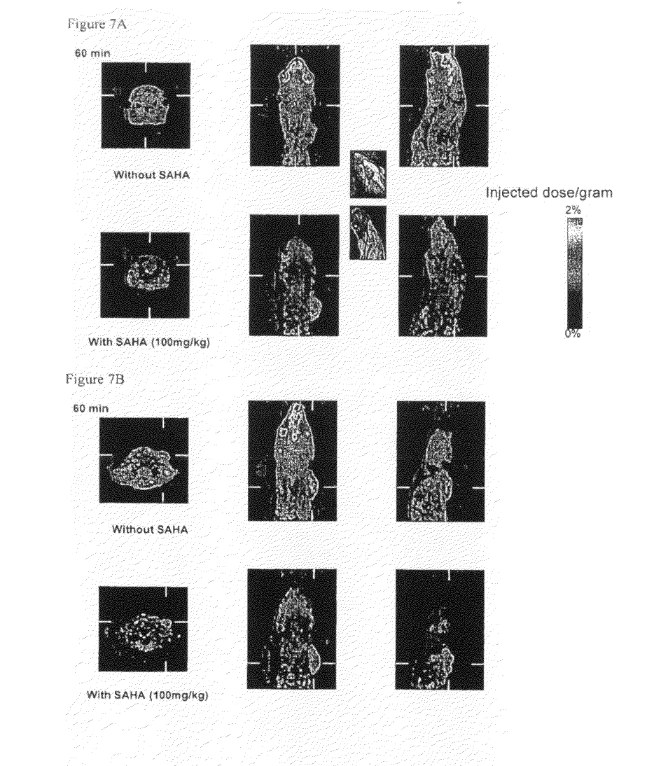

Results are shown in Table 1 and FIG. 5.

TABLE 1

| | Time (min) | | | | | |
|---|---|---|---|---|---|---|
| | 5 | 10 | 15 | 30 | 60 | 120 |
| Baseline | 1.95 +/− 0.13 | 2.09 +/− 0.15 | 2.11 +/− 0.12 | 2.02 +/− 0.11 | 1.84 +/− 0.14 | 1.38 +/− 0.12 |
| Inhibition | 1.16 +/− 0.03 | 1.16 +/− 0.01 | 1.16 +/− 0.03 | 1.16 +/− 0.04 | 1.15 +/− 0.01 | 1.13 +/− 0.02 |

### Example 4

### Assessment of HDAC Activity in Human Breast Carcinoma-Bearing Rats Using [¹⁸F]-FAHA and Positron Emission Tomography (PET)

Purpose: The aim of this study was to assess PET imaging of 6-([¹⁸F]-fluoroacetamide)-1-hexanoicanilide ([¹⁸F]-FAHA) for measuring histone deacetylase (HDAC) activity. HDAC plays an important role in regulation of gene expression inside tumor cells, including involvement in epigenetic rearrangement of chromatin architecture, which is a key event in maintenance of nuclear homeostasis and gene expression.

Methods: [¹⁸F]-FAHA was synthesized with high specific activity according to a method developed at M. D. Anderson Cancer Center. Human breast cancer cells, cell line MB435, grown as described in Example 3, were used to grow tumor xenografts in nude rats. Ten million cancer cells were injected subcutaneously in the neck area of six nude rats. When tumors were 1 cm in diameter, animals were anesthetized, injected with [¹⁸F]-FAHA (37 MBq) and PET imaging was performed (dynamic) up to 60 min post-injection. Two days after the initial imaging, another PET imaging with [¹⁸F]-FAHA was performed using a HDAC substrate (SAHA). The animals were given SAHA (50 mg/kg) intraperitoneally 1 hour prior to injection of the radiotracer.

Results: Tumor uptake and tumor-to-muscle (T/M) ratios of [¹⁸F]-FAHA are summarized in Table 2. During 60 min post-injection, the tumor uptake was increased, and achieved approximately 0.8% injected dose (ID)/g. The tumor-to muscle ratio reached 1.95. On the other hand, in the blocking study (treating with SAHA), the uptake inside tumor was significantly inhibited (p<0.01; unpaired t test).

TABLE 2

| | Without SAHA | With SAHA |
|---|---|---|
| % ID/g | | |
| 30 min | 0.78 ± 0.13 | 0.70 ± 0.04 |
| 60 min | 0.79 ± 0.13 | 0.71 ± 0.02 |
| T/M ratio | | |
| 30 min | 1.80 ± 0.17 | 1.52 ± 0.13 |
| 60 min | 1.95 ± 0.19 | 1.64 ± 0.10 |

Conclusions: This study suggests that [¹⁸F]-FAHA is a substrate of HDAC and can be used as a novel radiotracer for in vivo assessment of HDAC activity in tumor. PET imaging using this radiotracer could be a promising method for assessment of HDAC activity inside tumor cells, and has a potential for prediction of the effect of treatment with SAHA.

### Example 5

### In Vivo Assessment of HDAC Activity in Human Breast Carcinoma-Bearing Rats Using [¹⁸F]-FAHA and Positron Emission Tomography (PET)

MB435 human breast carcinoma cells were grown as described in Example 3, above. About 10×10⁶ MB435 cells were injected subcutaneously in female nu/nu rat. See Figure bbb. When tumor size was 15 mm in diameter, PET imaging studies with [¹⁸F]-FAHA were conducted in five rats using microPET R4 (Concorde). The animals were injected intravenously with 3.7 MBq (1mCi) of [¹⁸F]-FAHA and dynamic scanning (0-60 min) was performed. During the imaging session the rats were anesthetized with 2.0 vol % isoflurane/oxygen inhalation and continuously heated with a heating lamp. See FIG. 6.

Two days after the initial study, another [¹⁸F]-FAHA PET imaging with an substrate (SAHA) was performed. The animals were given SAHA (100 mg/kg) intraperitoneally 1 hour prior to injection of the [¹⁸F]-FAHA.

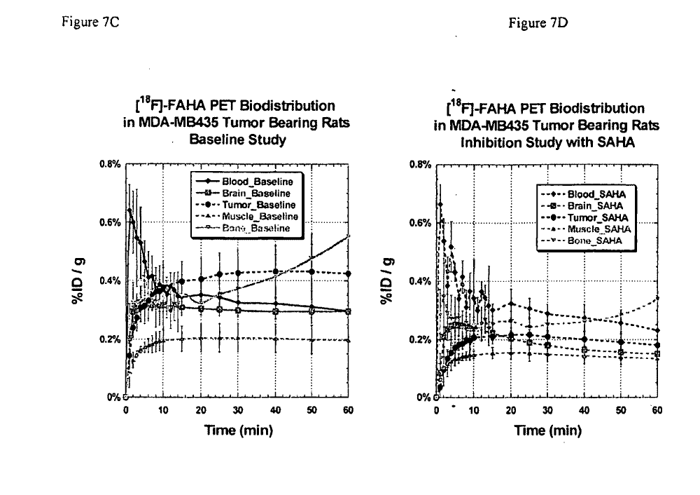

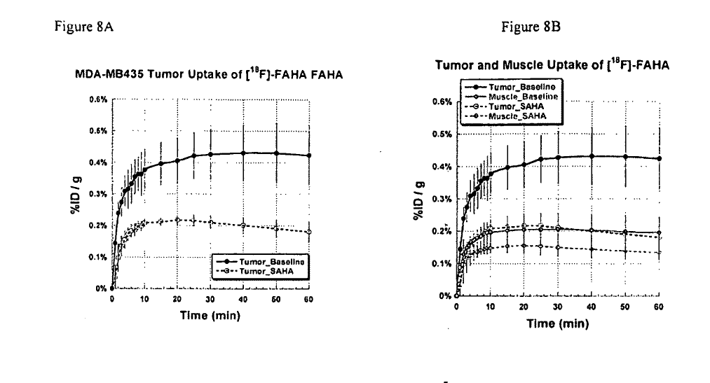

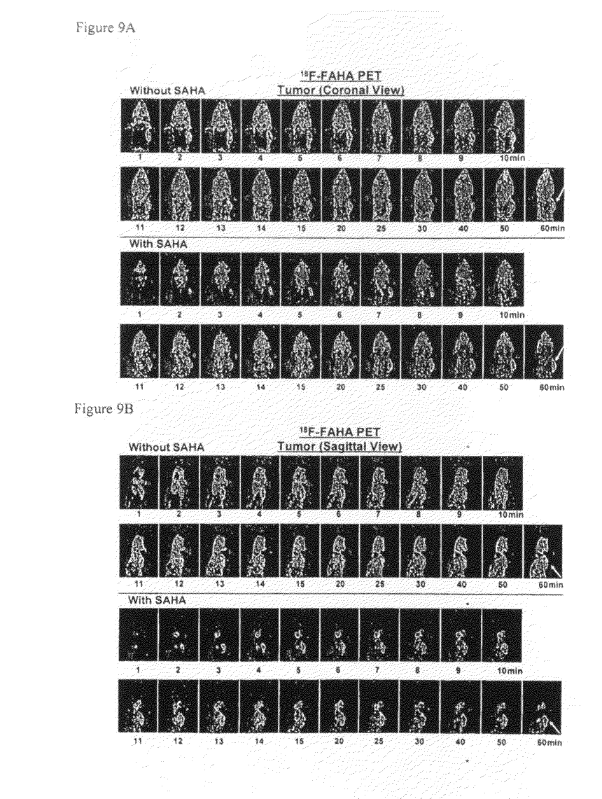

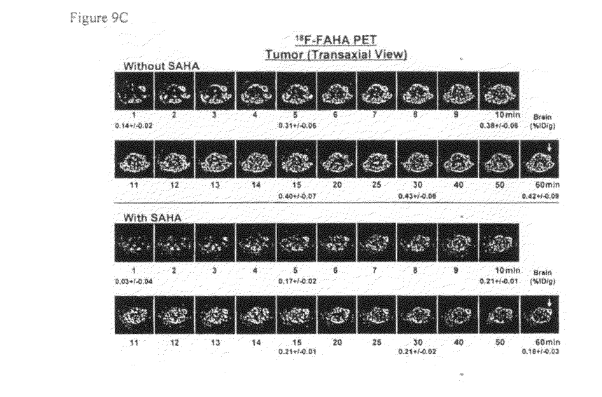

FIG. 7 shows PET images of exemplary rats and graphs of [¹⁸F]-FAHA uptake by various organs. FIG. 8 shows graphs of [¹⁸F]-FAHA uptake by the tumor and by muscle. Tumor-to-muscle uptake ratios are reported in Table 3.

TABLE 3

| | Time (min) | | | | |
|---|---|---|---|---|---|
| | 5 | 10 | 15 | 30 | 60 |
| Baseline | 2.01 +/− 0.57 | 2.05 +/− 0.76 | 2.08 +/− 0.83 | 2.20 +/− 0.95 | 2.38 +/− 1.11 |
| Inhibition | 1.43 +/− 0.15 | 1.50 +/− 0.15 | 1.46 +/− 0.16 | 1.47 +/− 0.12 | 1.47 +/− 0.11 |

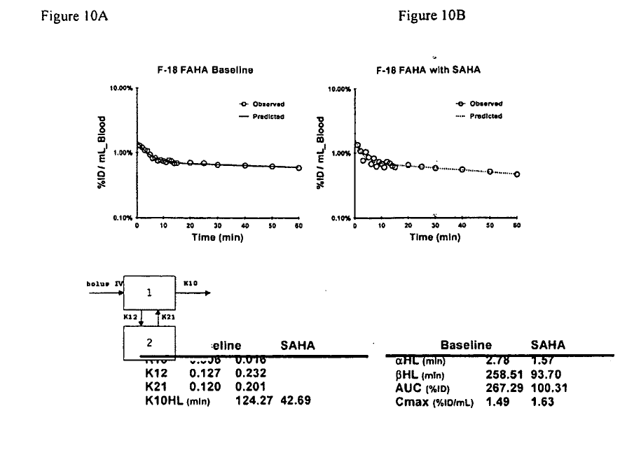

FIG. 9 shows a time course of [¹⁸F]-FAHA PET images of the tumor in exemplary rats.

FIG. 10 shows observed and predicted [¹⁸F]-FAHA levels in blood. The data was fit for pharmacokinetic modeling to get rate constants using a two compartmental analysis, and the observed data agreed with those predicted from the kinetic modeling. Therefore two compartment modeling fits with the observed values (% ID/mL Blood).

Both a Gjedde-Patlak plot and a Logan plot were taken. The Gjedde-Patlak plot shows irreversible binding as indicated by Kᵢ (influx rate constant). The linearity of the plot was checked and a plasma/reference region used as an input. The Logan plot shows reversible binding as indicated by DV, DVR and Bp. The linearity of the plot was checked and a plasma/reference region used as an input.

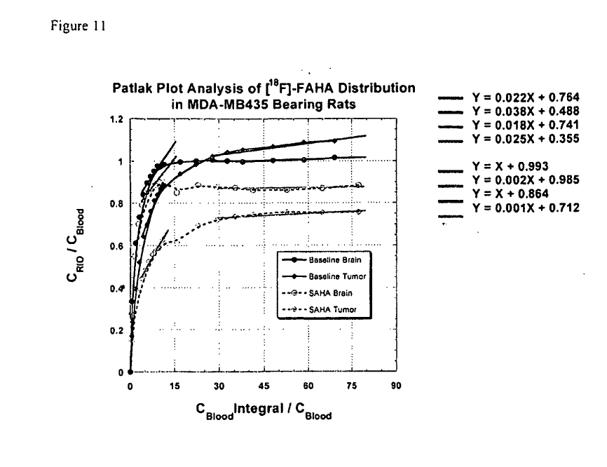

The Gjedde-Patlak plot result for the tumor is shown in FIG. 11 and Table 4. In Table 4, “baseline” refers to tumor uptake of FAHA without blocking by SAHA.

TABLE 4
Tumor

| | Baseline | with SAHA |
|---|---|---|
| Kᵢ 5-10 min (min⁻¹) | 0.038 | 0.025 |
| Kᵢ 30-60 min (min⁻¹) | 0.002 | 0.001 |

Kᵢ 5-10 min and Ki 30-60 min indicate behaviors of [¹⁸F]-FAHA and its metabolite, [¹⁸F]-FAc, respectively. Intraperitoneal injection of SAHA (100 mg/kg) affected partial inhibition on HDAC activity and lowered Kᵢ in the tumor.

The difference between Kᵢ 30-60 min in tumor and Kᵢ 30-60 min tumor with SAHA is considered the difference of amount of presented [¹⁸F]-FAc after partial inhibition of HDAC activity by SAHA, which reduced the production of [¹⁸F]-FAc from [¹⁸F]-FAHA inside cells.

### Example 6

### PET Imaging of HDAC Activity in Rat Brain Using 6-([¹⁸F]-fluoroacetamide)-1-hexanoicanilide ([¹⁸F]-FAHA)

Purpose: Histone deacetylase (HDAC) plays an important role in regulation of gene expression, and an substrate of HDAC (SAHA) has been reported as a potential neuroprotective agent. The aim of this study is to assess PET imaging of 6-([¹⁸F]-fluoroacetamide)-1-hexanoicanilide ([¹⁸F]-FAHA) in rat brain for measuring HDAC activity.

Methods: [¹⁸F]-FAHA was synthesized according to the methods developed in our laboratory in high specific activity. Imaging studies with [¹⁸F]-FAHA were conducted in five rats using a PET scanner under isoflurane inhalation anesthesia. The animals were injected intravenously with 37 MBq of [¹⁸F]-FAHA and dynamic scanning PET (0-60 min) was performed. During the imaging session, the rats were anesthetized with 1-1.5 vol % isoflurane/oxygen inhalation and continuously heated with a heating lamp. Two days after the initial study, another PET imaging was performed with [¹⁸F]-FAHA using an substrate (SAHA). Each animal was given SAHA (50 mg/kg), intraperitoneally 1 hour prior to injection of the [¹⁸F]-FAHA.

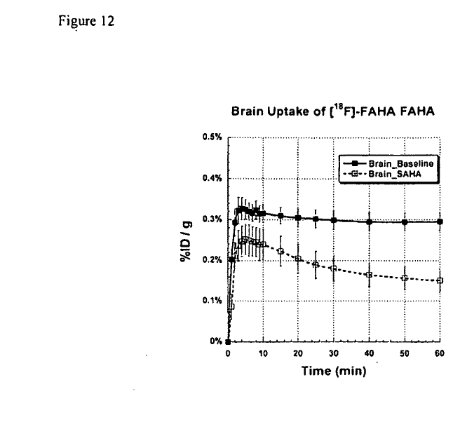

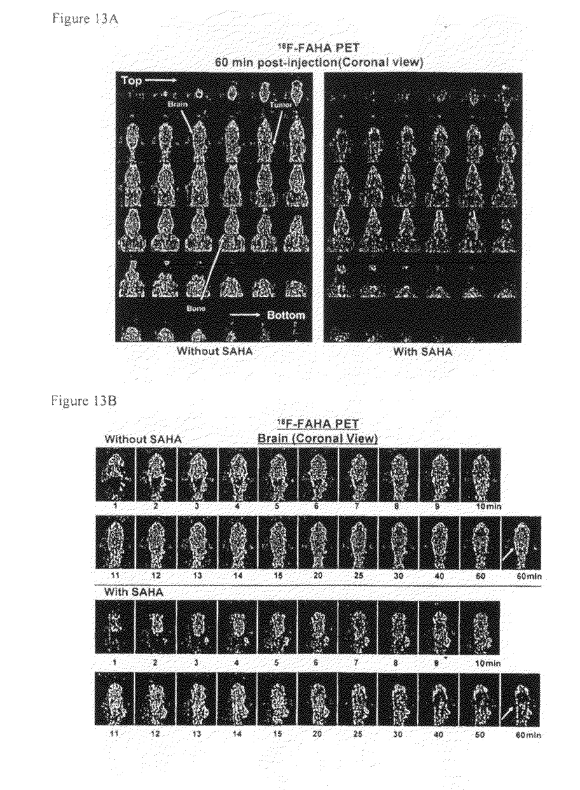

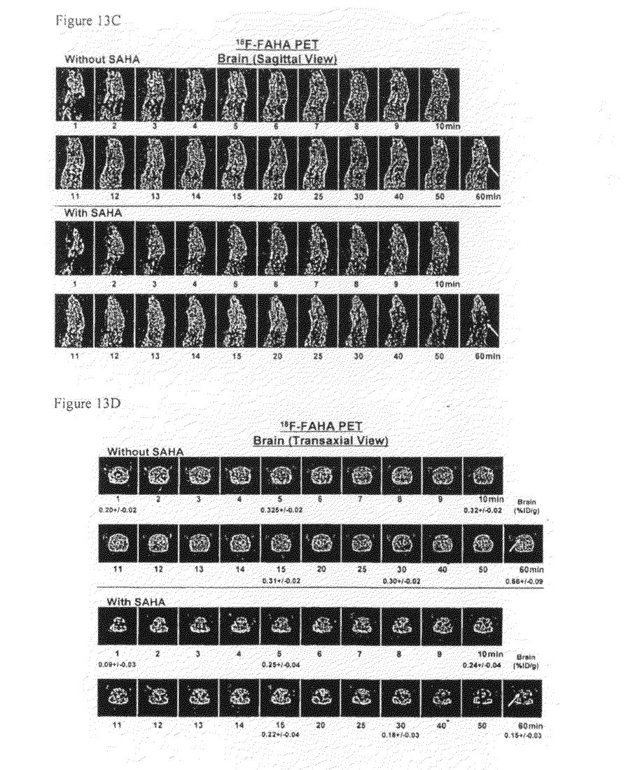

Results: Brain uptake and brain-to-muscle (B/M) ratios of [¹⁸F]-FAHA are summarized in Table 5 and FIG. 12. The brain uptake of this tracer increased rapidly and reached 0.44% injected dose/g. Also, the brain-to muscle ratio reached 1.95 at 5 min post-injection indicating presence of HDAC activity in brain. On the other hand, the uptake inside brain was significantly inhibited (p<0.01; unpaired t test) by SAHA.

TABLE 5

| | Without SAHA | With SAHA |
|---|---|---|
| % ID/g | | |
| 5 min | 0.44 ± 0.03 | 0.33 ± 0.05 |
| 60 min | 0.40 ± 0.03 | 0.23 ± 0.04 |
| B/M ratio | | |
| 5 min | 2.56 ± 0.33 | −1.52 ± 0.38 |
| 60 min | 2.10 ± 0.63 | 1.03 ± 0.08 |

Conclusions: This study suggests that this novel radiotracer, [¹⁸F]-FAHA, is a substrate of HDAC, and [¹⁸F]-FAHA may be a promising agent for assessment of HDAC activity in the brain. Furthermore, PET with [¹⁸F]-FAHA may be useful for the prediction of a therapeutic effect by treatment with SAHA.

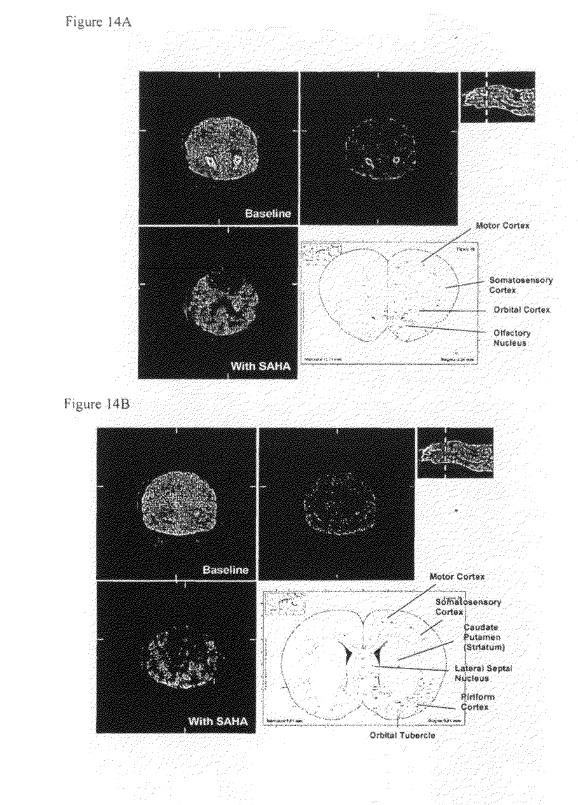

FIG. 13 shows a time course of [¹⁸F]-FAHA PET images of the rat brain.

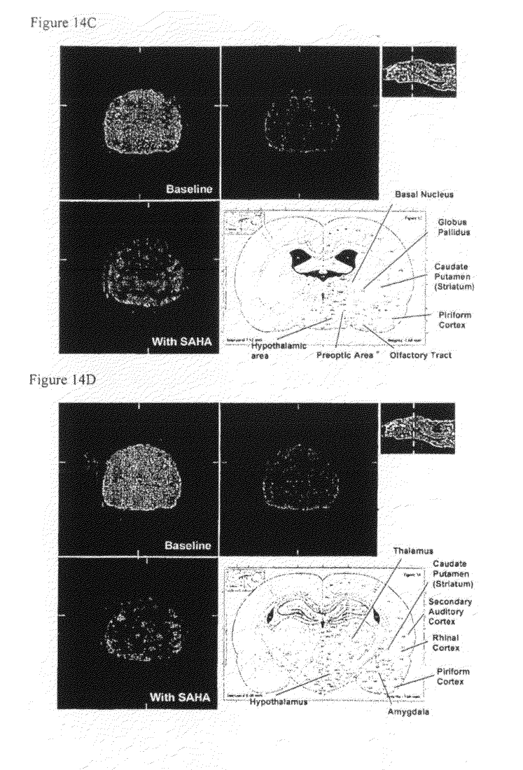

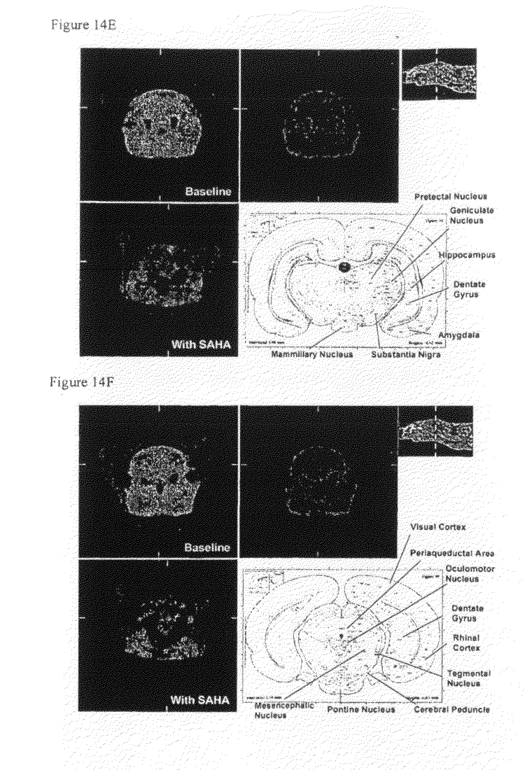

FIG. 14 shows transaxial sections of [¹⁸F]-FAHA PET images of the rat brain.

A Gjedde-Patlak analysis for the brain is shown in Table 6. In Table 6, “baseline” refers to brain uptake of FAHA without blocking by SAHA.

TABLE 6
Brain

| | Baseline | with SAHA |
|---|---|---|
| Kᵢ 5-10 min (min⁻¹) | 0.022 | 0.018 |
| kᵢ 30-60 min (min⁻¹) | >0.001 | >0.001 |

Kᵢ 5-10 min and Kᵢ 30-60 min indicate behaviors of [¹⁸F]-FAHA and its metabolite, [¹⁸F]-FAc, respectively. Intraperitoneal injection of SAHA (100 mg/kg) affected partial inhibition on HDAC activity and lowered Kᵢ in the brain.

Though not to be bound by theory, we consider it likely K, 30-60 min in the tumor is higher than that in the brain because [¹⁸F]-FAc itself can accumulate in the tumor cells, whereas [¹⁸F]-FAc is considered unlikely to enter the normal brain through the blood-brain barrier.

### Example 7

### Synthesis of Histone Deacetylase Substrates

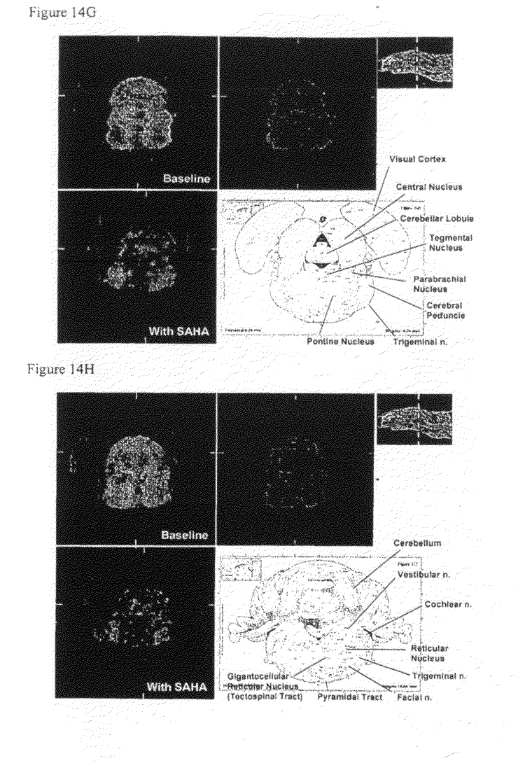

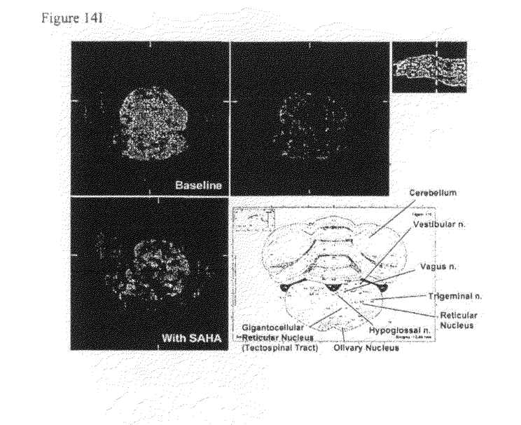

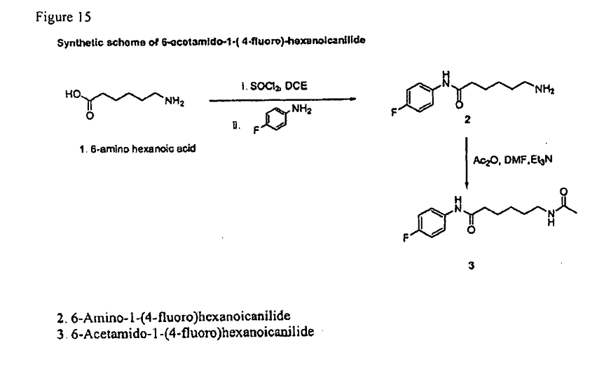

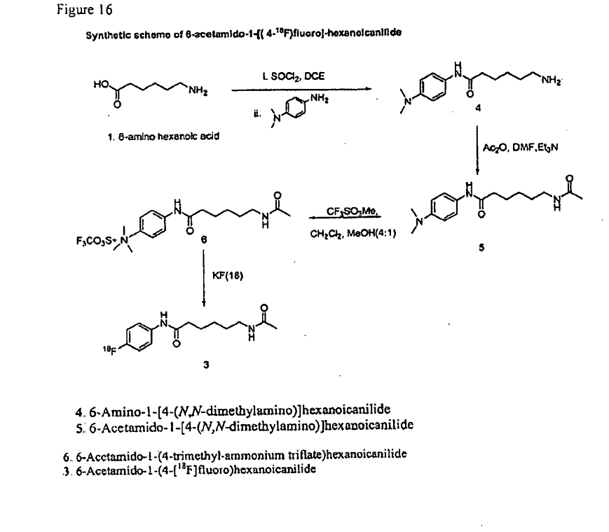

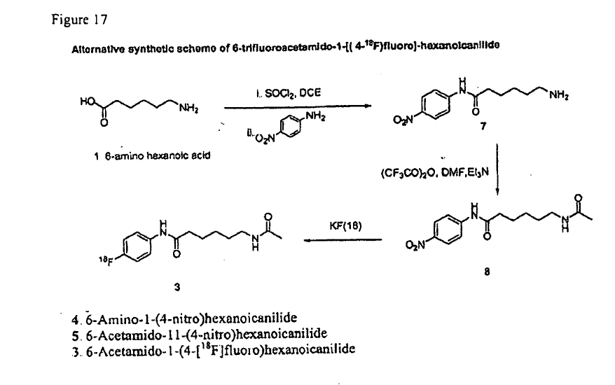

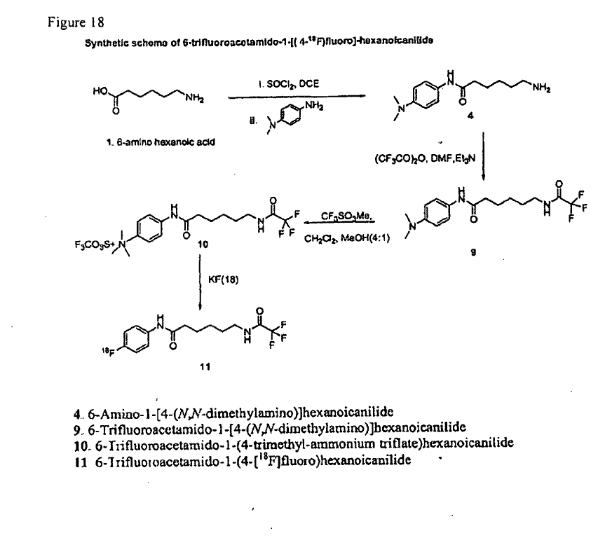

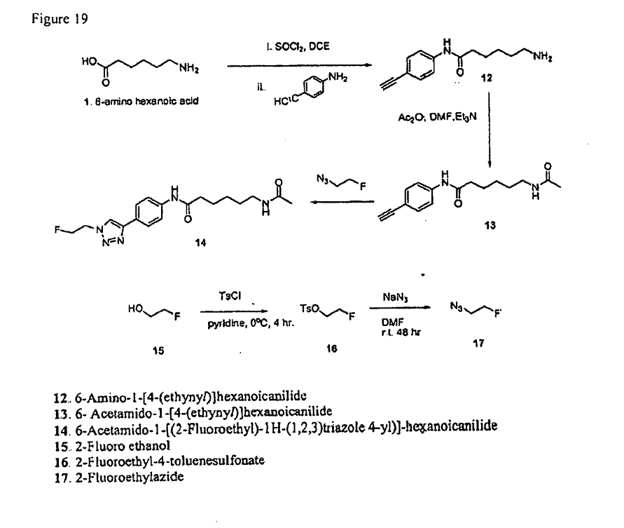

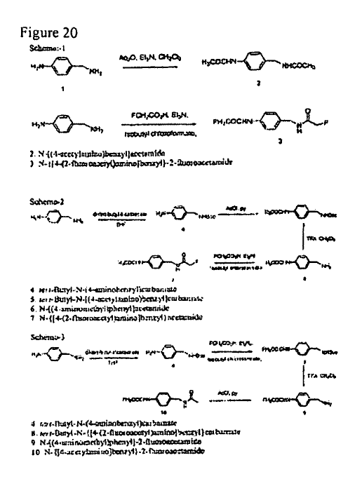

FIGS. 15-20 show synthesis schemes for various histone deactylase substrates according to various embodiments of the present invention.

### Example 8

### Histone Deacetylase and Sirtuin Activity in the Presence of Various Histone Deacetylase Substrates

HDAC Fluorimetric Assay

Material: Assay buffer, Assay developer, HDAC substrate

Source: BPS Bioscience, Inc.

Procedure:

Add reaction mixtures to low binding black plate. These include: HDAC assay buffer, Bovine serum albumin solution, HDAC substrate, and HDAC enzymes.

Incubate at 37° C. for 30 minutes.

Stop the reaction by addition of HDAC Assay developer (2×) and incubate plate at room temperature for 15 minutes.

Read sample in a microtiter-plate reading fluorimeter at excitation wavelength in the range of 350-380 nm and detect in emission range of 440-460 nm.

Sirtuin Fluorimetric Assay

Material: substrate available from manufacturer

Source: BPS Bioscience, Inc.

Procedure:

Add reaction mixtures to low binding black plate. These include: Sirtuin, HDAC assay buffer, Bovine serum albumin solution, NAD+ solution and Sirtuin substrate.

Incubate at 37° C. for 30 minutes

Stop the reaction by addition of HDAC Assay developer (2×) and incubate plate at room temperature for 15 minutes.

Read sample in a microtiter-plate reading fluorimeter at excitation wavelength in the range of 350-380 nm and detect in emission range of 440-460 nm.

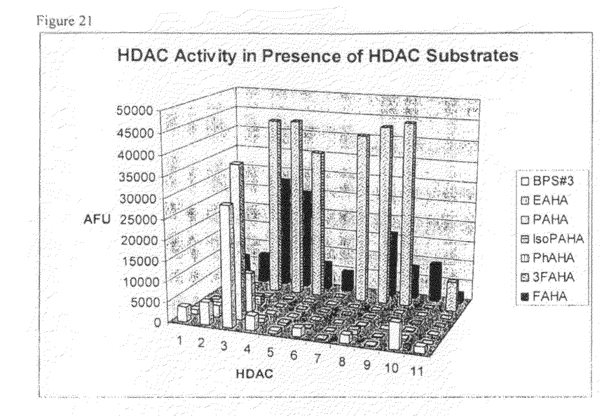

FIG. 21 shows histone deacetylase activity in the presence of histone deacetylase substrates EAHA, PAHA, IsoPAHA, PhAHA, 3FAHA, and FAHA. A known peptide, BPS#3, a fluorogenic, acetylated peptide substrate of HDACs (BPS Bioscience, San Diego, Calif.), was included as a negative control. As can be seen, PAHA, IsoPAHA, and PhAHA inhibited the activity of all HDACs.

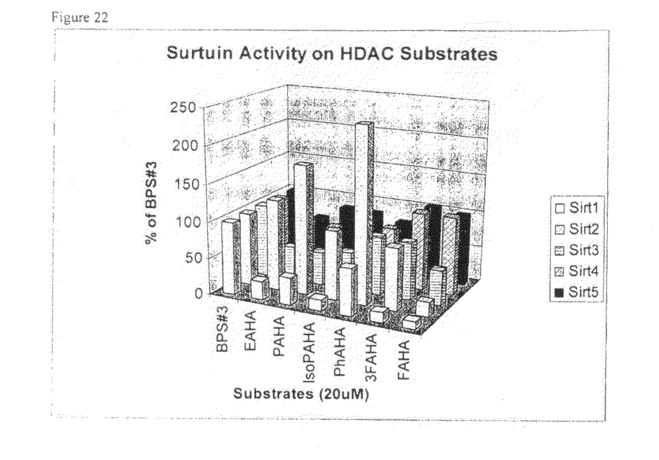

FIG. 22 shows sirtuin activity on 20 μM of each of the histone deacetylase substrates EAHA, PAHA, IsoPAHA, PhAHA, 3FAHA, and FAHA, relative to the enzyme activity on 20 μm BPS#3. As can be seen, all the tested HDAC substrates inhibited activity of sirtuin 1, and several of the HDAC substrates inhibited activity of sirtuins 2-5.

All of the compositions, methods, and apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions, methods, and apparatus and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Hamstra, D. A, Lee, K. C., Tychewicz, J. M., Schepkin, V. D., Moffat, B. A, Chen, M., Dornfeld, K. J., Lawrence, T. S., Chenevert, T. L., Ross, B. D., Gelovani, J. T., and Rehemtulla, A The use of 19F spectroscopy and diffusion-weighted MRI to evaluate differences in gene-dependent enzyme prodrug therapies. Mol Ther, 10: 916-928, 2004.
McSheehy, P. M., Leach, M. O., Judson, I. R, and Griffiths, J. R Metabolites of 2′-fluoro-2′ deoxy-D-glucose detected by 19F magnetic resonance spectroscopy in vivo predict response of murine RIF-1 tumors to 5-fluorouracil. Cancer Res, 60: 2122-2127, 2000.
Gade, T. P., Spees, W. M., Le, H. C., Zakian, K. L., Ponomarev, V., Doubrovin, M., Gelovani, J. G., and Koutcher, J. A. In vivo 5-fluorouracil and fluoronucleotide T1 relaxation time measurements using the variable nutation angle method. Magn Reson Med, 52: 169-173, 2004.
Chung, Y. L., Troy, H., Judson, I. R, Leek, R, Leach, M. O., Stubbs, M., Harris, A. L., and Griffiths, J. R Noninvasive measurements of capecitabine metabolism in bladder tumors overexpressing thymidine phosphorylase by fluorine-19 magnetic resonance spectroscopy. Clin Cancer Res, 10:3863-3870, 2004.
Aboagye, E. O., Artemov, D., Senter, P. D., and Bhujwalla, Z. M. Intratumoral conversion of 5-fluorocytosine to 5-fluorouracil by monoclonal antibody-cytosine deaminase conjugates: noninvasive detection of prodrug activation by magnetic resonance spectroscopy and spectroscopic imaging. Cancer Res, 58: 4075-4078, 1998:
Sankaranarayanapillai et al. Mol Cancer Ther. 2006 May; 5(5):1325-34)

What is claimed is:

1. A method of detecting a histone deacetylase activity in a mammal, comprising:
administering to the mammal a compound comprising at least one atom having a nucleus detectable by magnetic resonance spectroscopy, wherein the compound is a substrate of a histone deacetylase; and
observing the compound or a cleavage product thereof in at least a portion of the body of the mammal by magnetic resonance spectroscopy (MRS).

2. The method of claim 1, wherein the compound is selected from the group consisting of Boc-lysine trifluoroacetic acid (BLT); compounds having structure I: 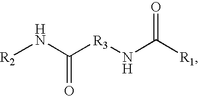
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -PhC≡C, -triazolyl-(CH₂)ₘX, -Ph-triazolyl-(CH₂)ₘX, -Ph(CH₂)ₘ, -Ph(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; and n is an integer from 0 to 10, inclusive; and salts and esters thereof.

3. The method of claim 1, wherein the mammal suffers a tumor in the portion of the body, and further comprising administering to the mammal a histone deacetylase substrate.

4. The method of claim 3, wherein the histone deacetylase substrate is selected from the group consisting of compounds having structure I: 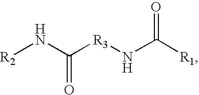
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -PhC≡C, -triazolyl-(CH₂)ₘX, -Ph-triazolyl-(CH₂)ₘX, -Ph(CH₂)ₘ(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙ alkyl; wherein each X is selected from the group consisting of —F and —Br; m is an integer from 1 to 5, inclusive; and n is an integer from 0 to 10, inclusive; and
salts and esters thereof.

5. The method of claim 4, wherein the pairing of the histone deacetylase and the histone deacetylase substrate is selected from the group consisting of HDAC 1 and 6-(fluoroacetamido)-1-hexanoicanilide (FAHA); HDAC-4 and FAHA; HDAC-5 and FAHA; HDAC-6 and FAHA; HDAC-8 and FAHA; HDAC-9 and FAHA; HDAC-4 and 6-(trifluoroacetamido)-1-hexanoicanilide (3FAHA); HDAC-5 and 3FAHA; HDAC-7 and 3FAHA; HDAC-8 and 3FAHA; HDAC-9 and 3FAHA; HDAC-11 and 3FAHA; HDAC-3 and 6-(methylacetamido)-1-hexanoicanilide (EAHA); sirtuin-2 and EAHA; HDAC-3 and 6-(ethylacetamido)-1-hexanoicanilide (PAHA); sirtuin-1 and PAHA; sirtuin-2 and PAHA; sirtuin-3 and PAHA; sirtuin-4 and PAHA; sirtuin-5 and PAHA; sirtuin-1 and 6-(isopropylacetamido)-1-hexanoicanilide (Iso-PAHA); sirtuin-2 and Iso-PAHA; sirtuin-3 and Iso-PAHA; sirtuin-4 and Iso-PAHA; sirtuin-5 and Iso-PAHA; sirtuin-1 and 6-(phenylacetamido)-1-hexanoicanilide (PhAHA); sirtuin-2 and PhAHA; sirtuin-3 and PhAHA; sirtuin-4 and PhAHA; and sirtuin-5 and PhAHA.

6. The method of claim 1, wherein the mammal suffers an insult in the brain, and further comprising administering to the mammal a histone deacetylase substrate.

7. The method of claim 6, wherein the histone deacetylase substrate is selected from the group consisting of compounds having structure I: 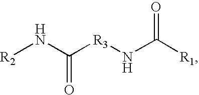
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -PhC≡C, -triazolyl-(CH₂)ₘX, -Ph-triazolyl-(CH₂)ₘX, -Ph(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙ alkyl; wherein each X is selected from the group consisting of —F and —Br; m is an integer from 1 to 5, inclusive; and n is an integer from 0 to 10, inclusive; and
salts and esters thereof.

8. The method of claim 7, wherein the pairing of the histone deacetylase and the histone deacetylase substrate is selected from the group consisting of HDAC 1 and 6-(fluoroacetamido)-1-hexanoicanilide (FAHA); HDAC-4 and FAHA; HDAC-5 and FAHA; HDAC-6 and FAHA; HDAC-8 and FAHA; HDAC-9 and FAHA; HDAC-4 and 6-(trifluoroacetamido)-1-hexanoicanilide (3FAHA); HDAC-5 and 3FAHA; HDAC-7 and 3FAHA; HDAC-8 and 3FAHA; HDAC-9 and 3FAHA; HDAC-11 and 3FAHA; HDAC-3 and 6-(methylacetamido)-1-hexanoicanilide (EAHA); sirtuin-2 and EAHA; HDAC-3 and 6-(ethylacetamido)-1-hexanoicanilide (PAHA); sirtuin-1 and PAHA; sirtuin-2 and PAHA; sirtuin-3 and PAHA; sirtuin-4 and PAHA; sirtuin-5 and PAHA; sirtuin-1 and 6-(isopropylacetamido)-1-hexanoicanilide (Iso-PAHA); sirtuin-2 and Iso-PAHA; sirtuin-3 and Iso-PAHA; sirtuin-4 and Iso-PAHA; sirtuin-5 and Iso-PAHA; sirtuin-1 and 6-(phenylacetamido)-1-hexanoicanilide (PhAHA); sirtuin-2 and PhAHA; sirtuin-3 and PhAHA; sirtuin-4 and PhAHA; and sirtuin-5 and PhAHA.

9. A method of detecting a histone deacetylase activity in a mammal, comprising:
administering to the mammal a compound comprising at least one positron-emission-decaying radioisotope, wherein the compound is a substrate of a histone deacetylase; and
observing the compound or a cleavage product thereof in at least a portion of the body of the mammal by positron emission tomography (PET).

10. The method of claim 9, wherein the compound is selected from the group consisting of compounds having structure I: 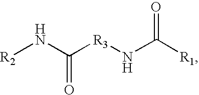
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, PhC≡C, -triazolyl-(CH₂)ₘX, -Ph-triazolyl-(CH₂)ₘX, -Ph(CH₂)ₘ, -Ph(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; n is an integer from 0 to 10, inclusive;
Boc-lysine tri[¹⁸F]fluoroacetic acid ([¹⁸F]BLT);
and salts and esters thereof.

11. The method of claim 10, wherein the pairing of the histone deacetylase and the histone deacetylase substrate is selected from the group consisting of HDAC 1 and 6-(fluoroacetamido)-1-hexanoicanilide (FAHA); HDAC-4 and FAHA; HDAC-5 and FAHA; HDAC-6 and FAHA; HDAC-8 and FAHA; HDAC-9 and FAHA; HDAC-4 and 6-(trifluoroacetamido)-1-hexanoicanilide (3FAHA); HDAC-5 and 3FAHA; HDAC-7 and 3FAHA; HDAC-8 and 3FAHA; HDAC-9 and 3FAHA; HDAC-11 and 3FAHA; HDAC-3 and 6-(methylacetamido)-1-hexanoicanilide (EAHA); sirtuin-2 and EAHA; HDAC-3 and 6-(ethylacetamido)-1-hexanoicanilide (PAHA); sirtuin-1 and PAHA; sirtuin-2 and PAHA; sirtuin-3 and PAHA; sirtuin-4 and PAHA; sirtuin-5 and PAHA; sirtuin-1 and 6-(isopropylacetamido)-1-hexanoicanilide (Iso-PAHA); sirtuin-2 and Iso-PAHA; sirtuin-3 and Iso-PAHA; sirtuin-4 and Iso-PAHA; sirtuin-5 and Iso-PAHA; sirtuin-1 and 6-(phenylacetamido)-1-hexanoicanilide (PhAHA); sirtuin-2 and PhAHA; sirtuin-3 and PhAHA; sirtuin-4 and PhAHA; and sirtuin-5 and PhAHA.

12. The method of claim 9, wherein the mammal suffers a tumor in the portion of the body, and further comprising administering to the mammal a histone deacetylase substrate.

13. The method of claim 12, wherein the histone deacetylase substrate is selected from the group consisting of compounds having structure I: 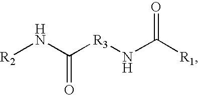
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, PhC≡C, -triazolyl-(CH₂)ₘX, -Ph-triazolyl-(CH₂)ₘX, -Ph(CH₂)ₘ, -Ph(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; and n is an integer from 0 to 10, inclusive; and
salts and esters thereof.

14. The method of claim 13, wherein the pairing of the histone deacetylase and the histone deacetylase substrate is selected from the group consisting of HDAC 1 and 6-(fluoroacetamido)-1-hexanoicanilide (FAHA); HDAC-4 and FAHA; HDAC-5 and FAHA; HDAC-6 and FAHA; HDAC-8 and FAHA; HDAC-9 and FAHA; HDAC-4 and 6-(trifluoroacetamido)-1-hexanoicanilide (3FAHA); HDAC-5 and 3FAHA; HDAC-7 and 3FAHA; HDAC-8 and 3FAHA; HDAC-9 and 3FAHA; HDAC-11 and 3FAHA; HDAC-3 and 6-(methylacetamido)-1-hexanoicanilide (EAHA); sirtuin-2 and EAHA; HDAC-3 and 6-(ethylacetamido)-1-hexanoicanilide (PAHA); sirtuin-1 and PAHA; sirtuin-2 and PAHA; sirtuin-3 and PAHA; sirtuin-4 and PAHA; sirtuin-5 and PAHA; sirtuin-1 and 6-(isopropylacetamido)-1-hexanoicanilide (Iso-PAHA); sirtuin-2 and Iso-PAHA; sirtuin-3 and Iso-PAHA; sirtuin-4 and Iso-PAHA; sirtuin-5 and Iso-PAHA; sirtuin-1 and 6-(phenylacetamido)-1-hexanoicanilide (PhAHA); sirtuin-2 and PhAHA; sirtuin-3 and PhAHA; sirtuin-4 and PhAHA; and sirtuin-5 and PhAHA.

15. The method of claim 9, wherein the mammal suffers an insult in the brain, and further comprising administering to the mammal a histone deacetylase substrate.

16. The method of claim 15, wherein the histone deacetylase substrate is selected from the group consisting of compounds having structure I: 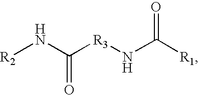
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -PhC≡C, -triazolyl-(CH₂)ₘX, -Ph-triazolyl-(CH₂)ₘX, -Ph(CH₂)ₘ(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; and n is an integer from 0 to 10, inclusive; and
salts and esters thereof.

17. The method of claim 16, wherein the pairing of the histone deacetylase and the histone deacetylase substrate is selected from the group consisting of HDAC 1 and 6-(fluoroacetamido)-1-hexanoicanilide (FAHA); HDAC-4 and FAHA; HDAC-5 and FAHA; HDAC-6 and FAHA; HDAC-8 and FAHA; HDAC-9 and FAHA; HDAC-4 and 6-(trifluoroacetamido)-1-hexanoicanilide (3FAHA); HDAC-5 and 3FAHA; HDAC-7 and 3FAHA; HDAC-8 and 3FAHA; HDAC-9 and 3FAHA; HDAC-11 and 3FAHA; HDAC-3 and 6-(methylacetamido)-1-hexanoicanilide (EAHA); sirtuin-2 and EAHA; HDAC-3 and 6-(ethylacetamido)-1-hexanoicanilide (PAHA); sirtuin-1 and PAHA; sirtuin-2 and PAHA; sirtuin-3 and PAHA; sirtuin-4 and PAHA; sirtuin-5 and PAHA; sirtuin-1 and 6-(isopropylacetamido)-1-hexanoicanilide (Iso-PAHA); sirtuin-2 and Iso-PAHA; sirtuin-3 and Iso-PAHA; sirtuin-4 and Iso-PAHA; sirtuin-5 and Iso-PAHA; sirtuin-1 and 6-(phenylacetamido)-1-hexanoicanilide (PhAHA); sirtuin-2 and PhAHA; sirtuin-3 and PhAHA; sirtuin-4 and PhAHA; and sirtuin-5 and PhAHA.

18. A histone deacetylase substrate, comprising:
a compound selected from the group consisting of compounds having structure I: 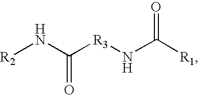
wherein R₁ is selected from the group consisting of —CH₃, —CH₂X, —CHX₂, —CX₃, —(CH₂)ₘCH₃, —CH₂(CH₃)₂, and -Ph; R₂ is selected from the group consisting of -Ph, -PhX, -PhN(CH₃)₂, -PhN⁺(CH₃)₃, -PhNO₂, -triazolyl-(CH₂)ₘX, -Ph-triazolyl-(CH₂)ₘX, -Ph(CH₂)ₘ(CH₂)ₘX, -Ph(CH₂)ₘN(CH₃)₂, -Ph(CH₂)ₘ, -PhN⁺(CH₃)₃, -Ph(CH₂)ₘNO₂, -Ph(CH₂)ₘ, -PhC≡C, -triazolyl-(CH₂)ₘX, -Ph-triazolyl-(CH₂)ₘX, -Ph(CH₂)ₘ(CH₃CONH), -Ph(CH₂)ₘ(CH₂XCONH), -Ph(CH₂)ₘ(CHX₂CONH), -Ph(CH₂)ₘ(CX₃CONH), —(CH═CH)(CH₂)ₘNH₂, -piperidinyl, and -piperidinyl-X; and R₃ is selected from the group consisting of —Cₙalkyl, -aryl, and -aryl-Cₙalkyl; wherein each X is selected from the group consisting of —F and —Br; m is an integer from 1 to 5, inclusive; and n is an integer from 0 to 10, inclusive; and
salts and esters thereof.

19. The histone deacetylase substrate of claim 18, wherein the compound comprises at least one radioisotope selected from the group consisting of ¹H, ²H, ³H, ¹¹C, ¹³C, ¹⁴C, ¹⁵N, ¹⁵O, ¹⁸F, ¹⁹F, and ³¹P.

20. The histone deacetylase substrate of claim 18, wherein the compound is selected from the group consisting of 6-(fluoroacetamido)-1-hexanoicanilide (FAHA), 6-(trifluoroacetamido)-1-hexanoicanilide (3FAHA), 6-(acetamido)-1-hexanoicanilide (AHA), 6-(methylacetamido)-1-hexanoicanilide (EAHA), 6-(ethylacetamido)-1-hexanoicanilide (PAHA), 6-(isopropylacetamido)-1-hexanoicanilide (Iso-PAHA), 6-(phenylacetamido)-1-hexanoicanilide (PhAHA), 6-(bromoacetamido)-1-hexanoicanilide (BrAHA), 6-(1-bromo-1-difluoroacetamido)-1-hexanoicanilide (Br2FAHA), 6-acetamido-1-[(1-ethyl(2-fluoro)piperidenyl-4-amino)]-hexanamide (FEPIAHA), 6-acetamido-1-[(2-fluoroethyl)-1H-(1, 2, 3)triazole-4-yl)]-hexanamide (FETrAHA), 6-acetamido-1-[piperidenyl-(4-amino)]-hexanamide (PIAHA), 6-(trifluoroacetamido)-1-(4-fluoro)hexanoicanilide (F—F3FAHA), 6-(1-bromo-1-difluoroacetamido)-1-(4-fluoro)hexanoicanilide (F—Br2FAHA), 6-acetamido-1-(4-fluoro)-hexanoicanilide, 6-acetamido-1-[4-(N,N-dimethylamino)]-hexanoicanilide, 6-acetamido-1-(4-trimethylammoniumtriflate)-hexanoicanilide, 6-acetamido-1-(4-nitro)-hexanoicanilide, 6-trifluoroacetamido-1-[4-(N,N-dimethylamino)]-hexanoicanilide, trifluoroacetamido-1-(4-trimethylammoniumtriflate)-hexanoicanilide, 6-acetamido-1-[4-(ethynyl]]-hexanoicanilide, 6-acetamido-1-[(2-fluoroethyl)-1H-(1,2,3)triazole-4-yl]]-hexanoicanilide, N-[(4-acetylamino)benzyl]acetamide, N-{[(4-(2-fluoroacetyl)amino)benzyl]}-2-fluoroacetamide, N-{[(4-(2-fluoroacetyl)amino)benzyl]}-acetamide, N-{[(4-acetylamino)benzyl]}-2-fluoroacetamide, N-{[(4-(2-bromooacetyl)amino)benzyl]}-2-fluoroacetamide, N-{[(4-(2-bromoacetyl)amino)benzyl]}-acetamide, and salts and esters thereof.
